(19) United States
(12) Patent Application Publication
(10) Pub. No.: US 2022/0287982 A1
(43) Pub. Date: Sep. 15, 2022

(54) EMBRYONIC MICROSPHERE PREPARATION METHOD AND PREPARATION MECHANISM, MICROSPHERE PREPARATION METHOD AND PREPARATION APPARATUS

(71) Applicant: BIODOSAGE TECH CO., LTD., Shanghai (CN)

(72) Inventor: TUO JIN, Shanghai (CN)

(73) Assignee: BIODOSAGE TECH CO., LTD., Shanghai (CN)

(21) Appl. No.: 17/633,471

(22) PCT Filed: Aug. 7, 2020
(86) PCT No.: PCT/CN2020/107670

§ 371 Date: Feb. 7, 2022

(30) Foreign Application Priority Data
Aug. 9, 2019 (CN) 201910736224.1

(51) Int. Cl. A61K 9/50; B01J 13/04

(52) U.S. Cl. CPC A61K 9/5089; B01J 13/04

(57) ABSTRACT

The invention discloses a method for preparing embryonic microspheres, a preparation mechanism, a method for preparing microspheres and a device for preparing the microspheres. The method for preparing the microspheres comprises delivering a microsphere-forming solution to a porous membrane located in a receiving liquid through a liquid transport member, to form embryonic microspheres; delivering embryo microspheres separated from the porous membrane along a channel filled with the receiving liquid, hardening the embryo microspheres to form microspheres; and collecting the microspheres. Wherein the flow rate of output liquid from the liquid transport member is controllable, so that an amount of output microsphere-forming solution per unit time is directly controlled, thereby regulating the particle size and uniformity of the generated microspheres. The present method improves the yield of the microspheres by eliminating variations in the surface tension distribution across the embryonic microspheres caused by mixing air bubbles into the embryo microspheres.

### CROSS-REFERENCE TO RELATED APPLICATIONS

This Application is a Section 371 National Stage Application No. PCT/CN2020/107670, filed on Aug. 7, 2020, and claims priority to Chinese Patent Application No. 201910736224.1, filed on Aug. 9, 2019, the contents of which are incorporated herein by reference in their entireties.

### FIELD OF THE INVENTION

The present invention relates to the field of microsphere preparation, in particular to a preparation method and preparation assembly of embryo microspheres, a microsphere preparation method and a preparation device.

### BACKGROUND OF THE INVENTION

Microspheres are tiny spherical particles, with diameters ranging from 1 to 250 μm. Polymer microspheres have great potential in the field of pharmaceutical science due to their good fluidity, ease of injection, and sustained release of the encapsulated ingredients, and have been extensively studied since the 1970s. The concept was first proposed R. Langer and J. Folkman in an article entitled “Polymers for sustained release of proteins and other macromolecules” published on Nature (263:793-800). In view of the fact that biological drugs are excellent in therapeutic efficacy but have to be administered by frequent injections due to their tissue membrane impermeability for oral dose, the authors suggested a sustained-release injection approach by encapsulating bio-medicines in biodegradable polymer microspheres.

Recombinant protein drugs grow rapidly at an annual rate of 14-16% since 1980s and have exceeded 50% of the global market of prescription drugs to date. There have more than 230 protein and peptide drugs approved for commercialization, and 9,000 are in the R&D pipeline, and some of these under-developing products may be launched to the market in the next few years. Contrast to the rapid growth of biological drugs, their administration is limited to frequent injection, and delivery technologies are waiting breakthroughs.

As an alternative to frequent injections, long-acting injections and efficient non-injection dosage forms are two conceivable solutions, which have attracted multi-decades-long R&D efforts of the scientists in the field. There has yet to be a breakthrough in non-injection biomedicine dosage form to date and have some long-acting injectives launched to the market. These market available bio-medicine injectives achieved long acting by chemical (PEGylation) or biological (sequence change or protein fusion) modification to prolong their in vivo half-life as well as by slow release at the injection site. The former may extend efficacy by only one or at most two weeks due to the exponential decay of their in vivo concentration; and moreover, their specific efficacy drops due to the hindrance effect of the modifying agents. The latter may theoretically maintain the efficacy of a single injection for weeks or even months, but it only succeeded in microsphere forms, and there are only 8 sustained-release microsphere products (excluding two contrast agents) thus far.

Why are there only limited biomedicines such as peptides, which are administrated by injection, formulated into microspheres, the only dosage forms feasible for several weeks long efficacy, despite these medicines are increasing? The critical hurdle is the cumbersome and poorly reproduce production process of microspheres. The current industrial process for producing microspheres includes two: double emulsification method and silicone oil phase separation method. The unit operations of the double emulsification method include emulsifying and dispersing the aqueous solution of peptides in an organic solution of a biodegradable polymer, further emulsifying, and dispersing the formed “water-in-oil” emulsion in the continuous phase of the polyvinyl alcohol aqueous solution to form a “complex emulsion”; and finally, evaporating the organic solvent under reduced pressure to solidify the polymer dispersed phase into spheres. This method has two distinct shortcomings: 1) The sizes of produced microspheres are diversified so that they have to be pre-lyophilized in order to sieve out under and over-sized microspheres under aseptic conditions. This process is cumbersome and inefficient for producing qualified microspheres; 2) As the inner aqueous phase of the double emulsion, the drug solution will inevitably contact the outer aqueous phase during emulsification and stirring, resulting in leakage and insufficient load of the drug in the microspheres. The uneven particle sizes and the drug leaking are highly sensitive to the shear force and duration of the stirring in the emulsification process, for which reproducible production is difficult to achieve. In order to avoid the uncontrollable drug leaking, phase separation method wherein silicone oil, which does not dissolve drugs is used as the continuous phase of the emulsification operation to ensure over 95% of the drug to be encapsulated in the microspheres. The silicone oil continuous phrase may also extract the organic solvent that dissolves the polymer, by which the polymer dispersed phase is solidified into spheres at the same time. Nevertheless, the issues of uneven particle sizes and low production yield remain. It is more troublesome, that the massive silicone oil used as the continuous phase has to be washed out with hydrocarbon solvents, the components of gasoline, which raises environmental production safety issues.

In order to solve the disadvantages of the methods discussed above, researchers in pharmaceutical technology have tried several improvement strategies, in which the methods named “microfluidizing” and “membrane-aided emulsification” are representative. The core step of microfluidizing is to inject the mixture of drug and polymer solutions dropwise from a nozzle into the flowing continuous phase during which the organic solvent is extracted, and the droplets are solidified, so that the drug is encapsulated in evenly sized particles. The fatal disadvantage of this method is its inefficiency. Such dropwise ejection process is only feasible for producing millimeter-sized spheres. When producing microspheres with a diameter a hundred times smaller, the production efficiency will be a million times lower (the volume is the third power of the diameter).

The key step of the membrane-aided emulsification method is to extrude the drug-loaded polymer solution through a cylindrical membrane made of porous materials by a compressed inert gas, by which the sizes of microspheres are adjusted by the pore diameter of the pre-made cylindrical membrane. The membrane emulsification method may improve the distribution of the particle size of the microspheres and encapsulation efficiency of water-soluble drugs. The droplets of the drug-loaded polymer solution (the so-called “embryonic microspheres”) may be extruded as snowflakes out of tens of thousands of membrane pores which ensures production efficiency. The membrane-aided emulsification method also suffers from a series of shortcomings, which limit its industrial application: 1) The embryonic microspheres departed from the membrane settle at the bottom of the container and may fuse into large particles, while as stirring for preventing their agglomeration may lead to breaking and fusion induced by shear force and collision, respectively. 2) The flow rate of the polymer solution driven by the compressed gas may be not linear to the pressure as it may be affected by factors such as the concentration, viscosity, and drug loading of the polymer solution, even room temperature; 3) Hydrophobic gas may have a considerable solubility in the organic solvent that dissolves the polymer, which causes some of the polymer droplets extruded to float up to the water surface and form flake shapes.

In view of this, the microfluidizing and membrane-aided emulsification methods for preparing microspheres have not resulted in feasible manufacture technology but remain at the stage of research and development stage despite the attempts have been reported in last decades.

To overcome the disadvantages of microfluidizing and membrane-aided emulsification methods, the inventor of the present invention previously disclosed a microsphere preparation process called “membrane-aided emulsification sedimentation method”, which combines membrane-aided emulsification and microfluidizing wherein the embryonic microspheres were solidified by extracting the solvents for the polymer during sedimentation to floating, followed by collection and rinsing. The membrane-aided emulsification sedimentation method solves one of the three problems of the membrane emulsification method, and other two challenges remain. To address the other two challenges, the present invention proposes a solution, precise injecting membrane-aided emulsification.

### SUMMARY OF THE INVENTION

To improve the yield of qualified products, the invention provides a method and preparation mechanism for preparing embryonic microspheres as well as a method and an apparatus for preparing microspheres, by which the size of the embryonic microspheres can be controlled precisely, and up-floating of the embryonic microspheres can be avoided.

The technological strategies provided by the present invention are described below.

The method for preparing embryonic microspheres comprises the following steps.

The microsphere-forming solution is transferred to the porous membrane which is placed in the receiving liquid using a liquid transport member, and the embryonic microspheres are formed by extrusion through the membrane holes; wherein, the flow rate of the output liquid from the liquid transport member is controllable.

In the present invention, the method for transferring the microsphere-forming solution is achieved using a flow rate controllable liquid transport member instead of the conventional compressed gas. Unlike the pressure gas driven method, wherein the gas pressure is only one of the controlling factors of the output rate of the microsphere forming solution, the liquid transport member may determine the amount of the microsphere forming solution output per unit time, which in turn determines diameters of microspheres formed. In addition, since the present invention eliminates the pressure gas driven process, the involvement of gas bubbles inside the embryonic microspheres which causes changed surface tension distribution and failure of sphere shape formation may be avoided.

Preferably, a syringe pump, a syringe, or other flow rate regulable pumps may be selected and used as the liquid transport member to transport the microsphere-forming solution to the porous membrane.

Preferably, shearing stress or vibration is applied to aid departure the embryonic microspheres from the porous membrane.

Wherein the intensity and/or frequency of applied shear or vibration can be controlled.

In the present invention, the applied frequency and intensity of shear force or vibration affect the departing rate of the microsphere forming solution from the surface of the porous membrane by changing sticking property of the embryonic microspheres on the surface of the porous membrane, by which the size of the formed embryonic microspheres is regulated.

Preferably, stirring, shaking, or other agitation actions are applied to the microsphere-forming solution in the liquid transport member.

In the present invention, when the microsphere-forming solution contains solid particles, the microsphere-forming solution in the liquid transport member is stirred, so that the particles do not settle and are evenly distributed during delivery.

A method for preparing microspheres, comprises the following steps.

S10, the microsphere-forming solution is transported to the porous membrane placed in the receiving liquid through the liquid transport member to form embryonic microspheres; wherein, the flow rate of the output liquid from the liquid transport member is controllable.

S20, the embryonic microspheres departing off the porous membrane flow along the channel filled with the receiving liquid, so that the organic solvent in the microsphere-forming solution is extracted, and the embryonic microspheres are hardened to microspheres.

S30, collecting the microspheres.

Preferably, in the step S10, the liquid transport member can be selected from a syringe pump, a syringe or other flow rate regulable pumps; and/or applying shearing stress or vibration to aid the embryonic microspheres detach from the porous membrane, and the intensity and frequency of applying shear or vibration can be adjusted; and/or: applying a stirring action to the microsphere-forming solution in the liquid transport member; and/or; degassing the equipment for preparing embryonic microspheres before transporting the microsphere-forming solution.

An embryonic microsphere preparation assembly comprises a liquid transport member for transporting a microsphere-forming solution at a controllable flow rate; a porous membrane for receiving the microsphere-forming solution from the liquid transport member and output it through micropores to form embryonic microspheres; the porous membrane holder is used to withhold the porous membrane and connect the liquid transport member and the porous membrane through its tubular structure.

In the present invention, the liquid transport member is used to replace the conventional gas pressure driven device and the container for microsphere-forming solution. Unlike the gas pressure driven design wherein the gas pressure is one of the influencing factors of the output rate of the microsphere-forming solution, the liquid transport member output microsphere-forming solution in a controllable rate. The liquid transport member can directly control the amount of the microsphere-forming solution output per unit time, and then can better control the particle size of the generated microspheres. In addition, since the assembly of the gas pressure driven device is replaced in the present invention, the problem of inability to form spheric shape resulted from gas bubbles up-taking into the embryonic microspheres to change the surface tension distribution is be avoided, and the yield of qualified product increases.

Preferably, a syringe pump, a syringe or other flow rate regulable pumps may be selected as the liquid transport member. To control the amount of the microsphere-forming solution output per unit time means, for example, the output amount/flow rate of the microsphere-forming solution is several milliliters or liters per second, as controlled or regulated by a syringe pump, syringe or other flow controllable pump.

Preferably, the liquid transport member comprises: a storage cavity for storing the microsphere-forming solution; a driving unit pushing the microsphere-forming solution along the inner wall of the storage cavity; and a power source for driving the pushing device.

Preferably, the bottom of the liquid transport member further comprises a stirring structure, and the stirring structure is used for stirring and agitating the microsphere-forming solution.

Preferably, a concave groove is formed at a bottom of the storage cavity to accommodate the stirring assembly.

Preferably, a raw material inlet and outlet are provided on the lower end sidewall of the storage cavity.

Preferably, it further comprises a feed pipe, the feed pipe connects the liquid transport member and the porous membrane holder, the porous membrane holder includes a tapered conical hole for withholding the feed pipe, the radial dimension of the tapered hole gradually increases in a direction from the inflow end to the outflow end of the microsphere-forming solution.

In the present invention, the design of the conical hole is for better sealing effect when the internal pressure becomes higher.

Preferably, an exhaust structure is provided on the porous membrane holder.

In the present invention, if gas exists in the porous membrane holder, the gas can be exhausted through the exhaust structure at this time.

Preferably, the feed pipe extends to near an entrance of the porous membrane.

In the present invention, the feeding pipe extends to near an entrance of the porous membrane, and the microsphere-forming solution can be directly transported to the porous membrane without introducing gas into the porous membrane, thereby affecting the yield of qualified embryonic microspheres.

A microsphere preparation apparatus comprises an embryonic microsphere preparation assembly; a solidification tube connected to the embryonic microsphere preparation assembly, wherein the embryonic microspheres settle in the solidification tube, then are solidified, and formed by solvent extraction to become microspheres; and a collector, connected with the solidification tube to collect the microspheres.

Preferably, a post-processing assembly is also included, and the post-processing assembly is used for removing organic solvents and other impurities from the microspheres.

To summarize, the present invention can achieve the following beneficial effects.

1. The pressure gas driven device and associated container for microsphere-forming solution were replaced by the liquid transport member. Unlike the pressure gas driving in which the gas pressure is only one of the factors affecting the output rate of the microsphere-forming solution, so that the output of microsphere-forming solution cannot be precisely controlled, while the liquid transport member output the microsphere-forming solution accurately. The sizes of the embryonic microspheres are related with the timing for them to detach from the porous membrane tube, which is determined by the growth rate of the embryonic microspheres, the surface tension, and the shear (or vibrational force) applied to the surface of the membrane tube. Among the above three factors, the growth rate of embryonic microspheres is determined by the flow rate of the microsphere-forming solution out of the membrane. Although the pressure of the driving gas affects the flow rate of the liquid, it does not necessarily ensure a linear relationship with the flow rate. The concentration and viscosity of microsphere forming solution and the amount of dissolved gas affect the relationship between gas pressure and flow rate. Using the flow rate of microsphere-forming solution to adjust the sizes of embryonic microspheres directly minimize the factors affecting particle sizes. Even for displaying accuracy, liquid flow rate is higher than gas pressure. These may greatly optimize the control of the size of the embryonic microsphere.

2. Replacing the conventional pressure gas driven method with the liquid transporting device reduces gas introduction in the process of forming embryonic microspheres, avoid the changes of surface tension distribution due to gas up taking into the embryonic microspheres, and improve the quality of microspheres.

3. By optimizing the output rate of the microsphere-forming solution from the liquid transport member, the shear force and vibration intensity or frequency, as well as the pore size of the porous membrane, the size of the microspheres can be precisely adjusted.

4. By the design of the concave groove at the bottom of the storage cavity of the liquid transport member, the microsphere-forming solution can be continuously stirred, so that its uniformity is well maintained during the process of liquid transportation, and equal quality of resulted embryonic microspheres is ensured.

### BRIEF DESCRIPTION OF THE DRAWINGS

With the drawings bellow, the preferred embodiments and the technical characteristics and advantages of the method for preparing the embryonic microsphere preparation discussed above will be described in an easily comprehensible manner.

FIG. 1 is a flow chart of the process for forming microspheres by the present invention;

FIG. 2 is a schematic description of the structure of the embryonic microsphere preparing assembly of the present invention;

FIG. 3 is a schematic description of the structure of an embodiment example of the embryonic microsphere preparing assembly;

FIG. 4 is a schematic description of the structure of another embodiment example of the embryonic microsphere preparing assembly;

FIG. 5 is a schematic description of a structure of the microsphere preparing assembly.

Reference elements in the drawings are: liquid transport member 1, storage cavity 101, pushing device 102, push rod 103, liquid discharge hole 104, connecting unit 105, stirring assembly 106, driving device 107, generating device 2, porous membrane holder 201, feed pipe 202, the exhaust structure 203, pressure gas retention chamber 204, the porous membrane 205, the solidification tube 3, the collecting container 4, and the conveying device 5.

### DETAILED DESCRIPTION OF THE PREFERRED EMBODIMENTS

In order to describe the embodiments or the technical solutions of the present invention more comprehensibly, some embodiments examples and some drawings will be referred in the discussions below. Obviously, these verbal and graphical description only represent some embodiment examples of the present invention. Ordinarily skilled technicians in the field may figure out alternative drawing and implementations on the basis of the above descriptions without creative efforts.

For the sake of brevity, the drawings shown below provide the related parts only which do not represent the whole actual structure of a product. In addition, for concise and easy understanding, one drawing is used to represent multiple devices or parts of similar functions schematically. Therefore, “one” does not necessarily means “only one”, but also “more than one” in this text.

The present invention provides an embodiment of a method for preparing embryonic microspheres, comprising the steps described below.

Embryonic microspheres are formed by transporting a microsphere-forming solution into the porous membrane placed in a receiving liquid through a liquid transport member and extruding the solution out of the membrane; wherein, the flow rate of the output liquid from the liquid transport member is controllable. The microsphere-forming solution is transported to the porous membrane through the liquid transport member 1, so that the solution can be extruded through the porous membrane to form designed shape.

The flow rate out of the liquid transport member is adjusted by choosing one or more of the following control methods:

a. Using a constant output flow rate to determine output the amount microsphere-forming solution per unit time.

b. Varying output flow rate of the microsphere-forming solution and parameters to meet the needs under different working conditions and requirements.

c. Pre-setting an output flow rate of the microsphere-forming solution to a not constant but a waved or stepwise manner according to the needs of different working conditions and requirements.

The liquid transport member enables the microsphere-forming solution to reach the porous membrane at a controllable flow rate to achieve the purpose of forming embryonic microspheres of controllable sizes. A syringe pump, a syringe, or other flow rate regulable pumps may be used to form the liquid transport member to push the microsphere-forming solution to the porous membrane. When a syringe pump is used to transport the solution, the flow rate may be set as constant, variable, as well as gradually increasing or decreasing rate; while the syringe pump may be manually operated or driven by a push assembly. The flow rate output from the syringe may be set as gradually increasing or decreasing, or constant. This embodiment does not limit flow rate and the specific structure of the liquid transport member 1. It should be noted that more transporting devices capable to transfer liquids with controllable flow rate can be used, in the present invention other than the two types listed in this embodiment.

Optionally, while the microsphere-forming solution is transported by the liquid transport member to the porous membrane placed in the microsphere-receiving liquid, shear force stress or vibration may be applied to facilitate the embryonic microspheres detach from the porous membrane. The vibrator may vibrate the microsphere-forming material to help the formed polymer droplets formed by through membrane extruding to detach from the surface of the porous membrane by dissociating the adhesion of the microsphere droplets on the. The vibrator may be driven by a pneumatic pusher, rods, electric push rods, manual push rods or any other form of reciprocating assembly. The intensity and frequency of the vibration can be adjusted at the same time or alternatively to achieve efficient production of even-sized microspheres.

Optionally, a stirring action is applied to the microsphere-forming solution in the liquid transport member 1 when the liquid is output.

Prior to transporting the microsphere-forming solution, the apparatus for preparing embryonic microspheres is exhausted/degassed, and the microsphere-forming solution may flow smoothly and easily through the porous membrane.

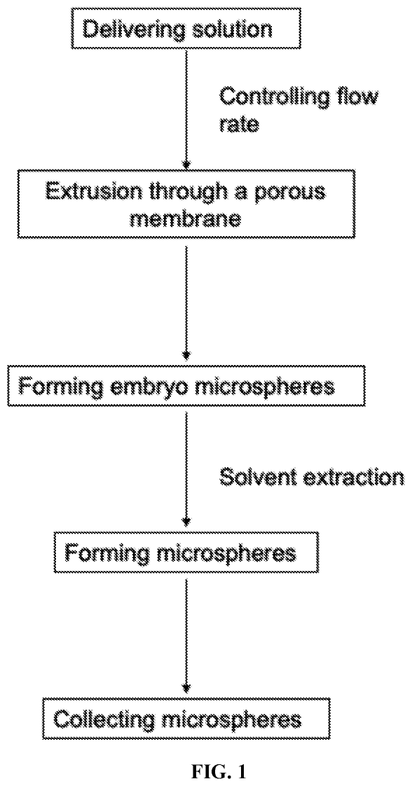

FIG. 1. Schematic diagram of one embodiment of the microsphere preparation method, while as the method comprises the following steps.

S10, the microsphere-forming solution is transported to the porous membrane placed in the receiving liquid through the liquid transport member to form embryonic microspheres; wherein, the flow rate of the output liquid from the liquid transport member is controllable.

S20, the embryonic microspheres falling off the porous membrane flow along the channel filled with the receiving liquid, during which the organic solvent in the microsphere-forming solution is extracted, and the embryonic microspheres are hardened to form microspheres.

S30, collecting microspheres.

In this embodiment, the microsphere-forming solution is output from the liquid transport member and reaches the porous membrane through the feed pipe. When the microsphere formation liquid is transported by the liquid transport member, the output amount of the microsphere formation liquid is controllable, and the size of the resulted embryonic microspheres may therefore be adjusted. The embryonic microspheres detached from the porous membrane flow along the channel filled with the receiving liquid, so that the embryonic microspheres turn to harden forms, and is collected by the subsequent collector.

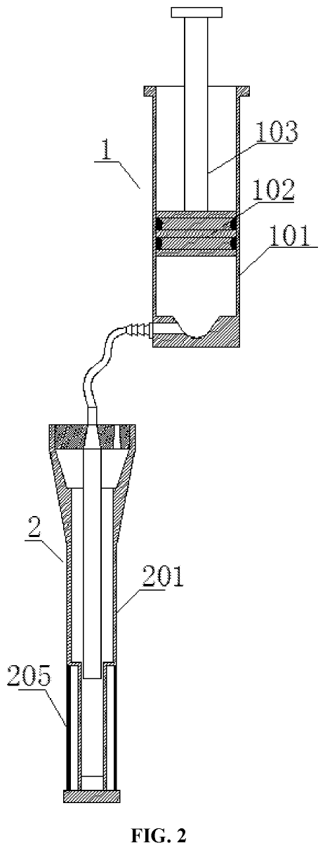

FIG. 2 is a schematic diagram of an embodiment of the embryonic microsphere preparation assembly. The embryonic microsphere preparation assembly includes: a liquid transport member 1, a porous membrane 205 and a porous membrane holder. The liquid transport member 1 is used for transporting the microsphere-forming solution at a controllable flow rate; the porous membrane 205 receives the microsphere-forming solution from the liquid transport member 1 and drives the solution to pass through the micropores to form embryonic microspheres; the porous membrane holder 201 is used for withholding the porous membrane 205 and connects the liquid transport member 1 and the porous membrane 205 by its tubular structure.

Optionally, a syringe pump, a syringe, or other flow rate regulable pumps, such as a metering pump, a molecular pump, a turbo pump, etc., can be used in the liquid transport member 1. In this embodiment, a syringe is used preferably for accurate flow rate of the microsphere forming solution and precise control of the size of the generated embryonic microspheres is controlled.

As referred in FIG. 2 again, the liquid transport member includes a storage cavity 101, a pushing unit 102 and a power source. The pushing unit 102 is slidably disposed along the inner wall of the storage cavity 101 for pushing the microsphere-forming solution; and a power source drives the pushing unit 102 to perform the pushing action. In practical operation, the pushing unit 102 is connected with the pushing rod 103, and then the power source drives the pushing rod 103 to drive the pushing unit 102 to slide, by which the pushing unit 102 is driven by the power source to move at a constant, as well as gradually increasing or decreasing flow rate, by which so that the flow rate of the microsphere-forming solution flowing to the porous membrane 205 is adjusted. The liquid transport member pumps the microsphere-forming solution to pass through the porous membrane 205 in a controlled flow rate, for example using a syringe pump, by which even-sized and less sticking particles are formed.

In this specific embodiment, the power source drives the pushing unit 102 to push the liquid in the storage cavity 101, and the pushing unit 102 slides along the inner wall of the storage cavity 101. During the specific operation, the power source may be manual or an external push assembly, such as hydraulic push, screw push. Options of pushing modes will not be described in detail in this embodiment.

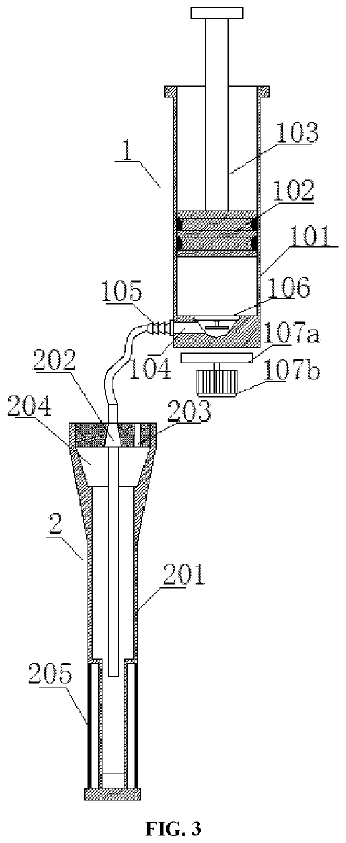

FIG. 3 is a schematic diagram of another embodiment of the embryonic microsphere preparation assembly. On the basis of the embodiments of the embryonic microsphere preparation assembly as referred in FIG. 2, the liquid transport member 1 may include a stirring unit 106, which is used for stirring the microsphere-forming solution; in the specific implementation. The paddle of stirring pr unit 106 may be driven magnetically at the bottom of the storage cavity 101 by an attached driving unit 107. Driving device 107 consists a motor 107a and magnet 107b, wherein the magnet is mounted on the rotor of motor 107a, and driven by motor 107a, by which it drives the paddle to rotate.

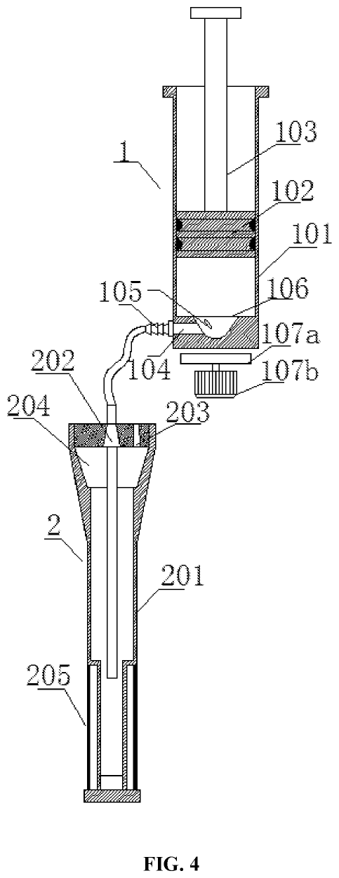

Optionally, as shown in FIG. 4, the stirring unit 106 can also be a magnetic stirring bar which is driven by driving unit 107 and stirs the microsphere-forming solution at the bottom of the storage cavity 101 in a better way.

Specifically, storage cavity 101 a concave groove at its bottom to accommodate a magnetic stirring unit 106. The concave groove enables the liquid to be better stirred when it passes the inner bottom of the storage cavity 101 for pushing unit 102 to push the liquid to flow. The assembly involving the concave groove and the stirring unit 106 ensures the microsphere-forming solution be stirred homogeneously without precipitation and be depleted with minimal leftover in side storage cavity 101.

Optionally, the storage cavity 101 is provided with inlet-outlet opening at its low side wall for raw materials to be transported in and out, by which the liquid may be filled in and pumped out alternatively during production operation without open-cavity refilling. Better working efficiency may therefore be achieved.

Optionally, as shown in FIGS. 3-4, the embryonic microsphere preparation assembly further includes a feed pipe 202, which connects between the liquid transport member 1 and the porous membrane holder 201. In the specific implementation, the liquid transport member 1 is provided with an outlet 104 on its side wall for firm connection to feed pipe 202 through a barb-shaped connecting piece 105 and transferring microsphere forming solution. The porous membrane holder 201 includes a tapered hole for withholding the feed pipe 202, and the radial size of the tapered hole gradually increases from the outside to the inside; the design of the tapered hole is to ensure the higher the internal pressure, the tighter the sealing effect. The feed pipe 202 extends to the porous membrane 205, and the liquid is directly transported to the porous membrane 205 with the feed pipe 202 to avoid locking the air into the porous membrane 205. Practically air bubbles in the porous membrane 205 will affect the quality and yield of microspheres production.

Optionally, the porous membrane holder 201 is provided with an exhaust structure 203, and the exhaust hole in which the exhaust structure 203 installed is tapered, which can have a sealing effect. The upper end is a gas retention cavity 204, which can be discharged through the exhaust structure 203 when the gas increases. Generating device 2 consists porous membrane holder 201, feed pipe 202, exhaust structure 203, gas retention cavity 204, and porous membrane 205. When the feed pipe 202 transports the microsphere-forming solution, specifically to near the entrance of the porous membrane, the mixed gas in the microsphere-forming solution will enter the gas retention cavity 204, and then be discharged through the exhaust structure 203 to avoid locking the gas in the porous membrane 205, and avoid the effect of gas on the particle size of embryonic microspheres during the production of embryonic microspheres.

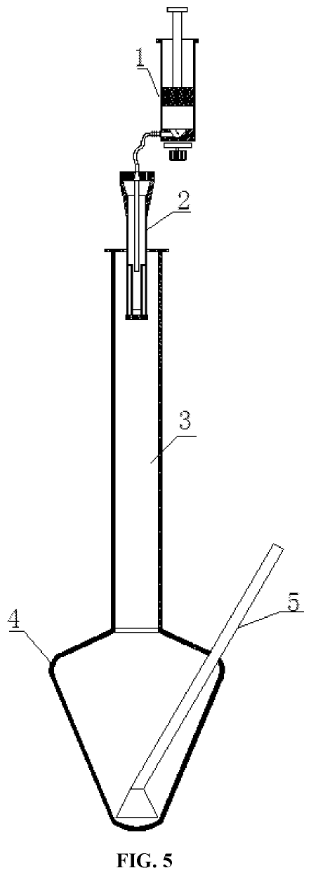

FIG. 5 is a schematic diagram of an embodiment of a microsphere preparation assembly. The microsphere preparation assembly includes: the embryonic microsphere preparation assembly discussed in any of the foregoing embodiments; a solidification tube 3 connected to the embryonic microsphere preparation assembly, wherein the embryonic microspheres settle and are solidified to microspheres due to solvent extraction to form microspheres; and the collector 4 connected with the solidification tube 3 wherein the microspheres are collected.

Specifically, the microsphere preparation assembly also includes a post-processing assembly, wherein organic solvents and other impurities are eliminated from the microspheres.

In this embodiment, the microsphere forming solution is converted into embryonic microspheres by using the embryonic microsphere preparation assembly, and the formed embryonic microspheres are solidified to microspheres by organic solvent extracting when they pass through the solidification tube 3. Then the microspheres are collected in the collector 4. At the same time, the generating device 2 takes a circular motion in the solidification tube 3 to create a shearing stress to facilitate the embryonic microspheres to fall off from the porous membrane 205. Then, the formed microspheres are transported to the post processing device through the conveying member 5, for post-processing. During the post processing, the microspheres are rinsed a freeze-dried hereafter.

In the foregoing embodiments, the description of each embodiment may have focused to each respective aspect. Some description or record may be insufficient in details in one embodiment, but the relevant detailed descriptions may be found in other embodiments.

It should be noted that the description of each of the above embodiments consists of the required technical aspects arbitrarily for providing examples for each preferred embodiments of the present invention. For those skilled in the art, improvements and modifications can be made on the basis of the present invention. These should be regarded to be within the protection scope of the present invention.

1. A method to prepare embryonic microsphere, comprising forming embryonic microspheres naturally in the absence of gas pressure transport, comprising
transporting a microsphere-forming solution to a porous membrane in the microsphere-receiving liquid through a liquid transport member, and forming embryonic microspheres by extrusion through porous membrane holes;
controlling a flow rate of an output liquid from the liquid transport member as an amount of the microsphere-forming solution output per unit time.

2. The method to prepare embryonic microspheres according to claim 1, wherein the liquid transport member is selected from a syringe pump, syringe or other flow controllable pump.

3. The method to prepare embryonic microspheres according to claim 1, wherein the embryonic microspheres are separated from the porous membrane by applying shear force or vibration, wherein, an intensity and/or frequency of the applied shear force or vibration is controllable.

4. The method to prepare embryonic microspheres according to claim 1, wherein stirring, shaking, or other agitation actions are applied to the microsphere-forming solution in the liquid transport member.

5. The method to prepare embryonic microspheres according to claim 1, wherein before transporting the microsphere-forming solution, the equipment for preparing embryonic microspheres is exhausted.

6. A method for preparing microspheres, comprising
S10, the microsphere-forming solution is transported to a porous membrane located in a receiving liquid through a liquid transport member to form embryonic microspheres;
wherein, a flow rate of an output liquid from the liquid transport member is controllable;
S20, the embryonic microspheres falling off the porous membrane flow along a channel filled with the receiving liquid, so that an organic solvent in the microsphere-forming solution is extracted, and the embryonic microspheres are hardened to form microspheres.
S30, collecting the microspheres.

7. The method for preparing microspheres according to claim 6, wherein in the step S10:
the liquid transport member is selected from a syringe pump, a syringe or other pump with adjustable flow; and/or;
applying shearing force or vibration to make the embryonic microspheres separate from the porous membrane, and intensity and frequency of the applied shearing force or vibration are controllable; and/or:
applying a stirring action to the microsphere-forming solution in the liquid trans member; and/or;
before transporting the microsphere -forming solution, vent the equipment for preparing embryonic microspheres.

8. An embryonic microsphere preparation assembly, comprising:
a liquid transport element, transporting microsphere-forming solution at a controllable flow rate;
a porous membrane, receiving the microsphere-forming solution from the liquid transport member and passing it through micropores to form embryonic microspheres; and
a porous membrane holder, withholding the porous membrane and connecting the liquid transport member and the porous membrane through its tubular structure.

9. The embryonic microsphere preparation assembly according to claim 8, wherein the liquid transport member is selected from a syringe pump, a syringe, or any other pump which has an adjustable flow rate.

10. The embryonic microsphere preparation assembly according to claim 8, wherein
the liquid transport member comprises a storage cavity, configured to store the microsphere-forming solution;
a pushing member, slidably disposed along an inner wall of the storage cavity, for pushing the microsphere-forming solution; and
a power source, driving the pushing member to perform a pushing action.

11. The embryonic microsphere preparation assembly according to claim 10, wherein
a bottom of the liquid transport member further comprises a stirring assembly, and the stirring structure is used to agitate the microsphere-forming solution.

12. The embryonic microsphere preparation assembly according to claim 8, wherein
a concave groove is formed at a bottom of the storage cavity to accommodate the stirring assembly.

13. The embryonic microsphere preparation assembly according to claim 8, wherein
a raw material inlet and outlet are provided on a lower end sidewall of the storage cavity.

14. The embryonic microsphere preparation assembly according to claim 8, further comprising
a feed pipe, the feed pipe is in liquid communication with the liquid transport member and the porous membrane holder, and the porous membrane holder includes a tapered hole for accommodating the feed pipe, and a radial dimension of the tapered hole gradually increases in a direction from the inflow end to the outflow end of the microsphere-forming solution outside to inside of the tapered hole.

15. The embryonic microsphere preparation assembly according to claim 8, wherein
an exhaust structure is provided on the porous membrane holder.

16. The embryonic microsphere assembly of claim 14, wherein
the feed pipe extends to the porous membrane.

17. An apparatus for preparing microspheres, comprising:
an embryonic microsphere preparation assembly according to claim 8;
a solidification tube, which is connected to the embryonic microsphere preparation assembly, and the embryonic microspheres precipitate in the solidification tube, and solidified to form microspheres by solvent extraction; and
a collector is connected to the solidification tube to collect the microspheres.

18. An The apparatus for preparing microspheres of claim 17, further comprising
a post-processing assembly for removing organic solvents and other impurities from the microspheres.
